(19) United States
(12) Patent Application Publication
(10) Pub. No.: US 2010/0286552 A1
(43) Pub. Date: Nov. 11, 2010

(54) WIPING IMPLEMENT PARTICULARLY USEFUL FOR DETERMINING WHETHER A WOMAN IS IN A NIDDAH STATUS

(76) Inventor: Miryam ABITBOL, Thornhill (CA)

Correspondence Address: MARTIN D. MOYNIHAN d/b/a PRTSI, INC., P.O. BOX 16446, ARLINGTON, VA, 22215, US

(21) Appl. No.: 12/774,804

(22) Filed: May 6, 2010

(30) Foreign Application Priority Data
May 7, 2009 (IL) 198655

(51) Int. Cl. A61B 10/00

(52) U.S. Cl. 600/551

(57) ABSTRACT

A wiping device for wiping the surface of a cavity, comprising an implement having a distal end dimensioned and configured with a rounded tip to facilitate insertion of the device into the cavity, the implement also having a proximal end configured for manually grasping the implement and for inserting the distal end of the implement into the cavity, and a flexible wiping cloth configured to be received over the distal end of the implement, to conform to its rounded tip, and to define a lower edge to be releasable engaged by said implement in order to be releasably retained on the implement.

### RELATED APPLICATION/S

This application claims the benefit of priority from Israel Patent Application No. 198655, filed on May 7, 2009, the contents of which are incorporated by reference as if fully set forth herein.

### FIELD AND BACKGROUND OF THE INVENTION

The present invention relates to wiping implements generally useful for wiping surfaces of a tubular passageway. The invention is particularly useful for determining whether a Jewish woman is in a Niddah status according to the Jewish laws of family purity, and the invention is therefore described below with respect to such an application.

The term Niddah is a Hebrew term which literally means “separation”, and generally refers to separation from ritual impurity. Niddah is the main category of Jewish law concerning sexual matters—also referred to as family purity (Hebrew term being Taharat haMishpacha).

A Jewish woman is said to be in the Niddah status, according to orthodox rabbinical law, when the woman is menstruating or has menstruated without yet completing the associated ritual requirements for “Tahara”.

The biblical regulations of Leviticus (15:19-30; 18:19; and 20:18) specify that a menstruating woman has to be separated from other people for seven days. Anything she comes into contact with would become ritually impure during this period, and anyone who comes into contact with these things, or her, during this period would also become ritually impure, until the evening came and the persons making contact had washed themselves and their clothes in water.

According to orthodox rabbinical law, a woman becomes a Niddah when she is aware that blood has come from her womb (or uterine), whether it is due to menstruation, childbirth, sexually transmitted disease, or other reasons. Even if menstruation started before she sees evidence of the flow of blood, the rabbinical regulations regard her as not being a Niddah until she notices the blood. Until this point the regulations do not come into force.

It is not necessary for the woman to witness the flow of blood itself, and it is sufficient for her to notice a stain that has indications of coming from her womb. Blood stains are inadequate without such evidence; for example, if she finds a stain just after cutting her finger, she does not become a Niddah, as the blood is not obviously from the womb. If there is a blood stain of uncertain origin, for example on her underclothing, there are a series of complicated criteria given by rabbinical law to determine whether she is a Niddah or not; the woman herself is not expected to know these criteria, and can seek the assistance of a rabbi who is sufficiently learned in them.

The Niddah state lasts at least 12 days in the Ashkenazic—the 5 days minimum and the subsequent seven days. The count of days begins when the woman first sees her menstrual blood, and ends 12 days later, or 7 days after the menstruation ceases (whichever is longer). For non-Ashkenazic Jewry there are a variety of customs. Although this count could start in the middle of the day, it is always considered to end on the evening of the final day. Most Sephardic Jews use a slightly more lenient calculation resulting in a minimum of 11 days.

In the Orthodox Jewish community, women may test whether menstruation has ceased; this ritual is known as the “hefsek tahara”. The woman takes a bath or shower near sunset, wraps a special cloth around her fingers, and swipes the vaginal circumference. If the cloth only contains discharges that are white, yellow, or clear, then the menstruation is considered to have ceased. If discharge is bright red then it indicates that menstruation continues. If it is any other color it is subject to further inquiry, often involving the consultation of a rabbi. The ritual requires that the cloth used to perform this test is first checked carefully to ensure that it is clean of any marks, colored threads, or specks; the cloth itself can be any clean white cloth, although there are small cloths designed for this ritual, known as “bedikah” (meaning checking). There are further rituals for giving assurance about the ceasing of menstruation. After the “hefsek tahara”, some women insert a cloth (or, in modern times, a tampon), to ensure that there is no uterine blood; this must be done carefully, as it could otherwise irritate the membrane and cause bleeding unrelated to menstruation.

The “bedikah” cloth, called an “eid” in Hebrew is a clean piece of white cloth used in the process of purifying a Niddah. It is used by a Jewish woman to determine whether she has finished menstruation. The cloth is inserted into the vagina, and if no blood is found, the woman may start counting for blood-free days. On each of these days, she performs the examination in the morning and in the late afternoon. If no blood is found, she may to the “mikveh” (ritual bath) on the seventh evening, and then engage in relation with her husband.

Such cloths are about 2×4 inches and are available at local Judaica stores, the local “mikveh”, or may be cut from clean off-white soft cotton cloth.

After the Niddah state is over, the woman is to go to a special type of bath, known as a mikvah, to simplify ritual washing, although certain forms of immersion in natural streams, lakes, and even the sea, if cleared by a rabbi, are still considered sufficient. According to tradition, there must be nothing between the woman and the water at any point of her body, and therefore before bathing, the woman is traditionally required to remove all jewelry, make-up, and any other obstructions (defined in such a way that in modern times this would include contact lenses); the rabbinical tradition requires full immersion, including the whole of the hair.

### OBJECTS AND BRIEF SUMMARY OF THE PRESENT INVENTION

One object of the present invention is to provide a wiping implement which is useful generally for wiping the surfaces of a tubular passageway. A more particular object of the invention is to provide an implement to aid in determining whether a woman is in the Niddah status. A further object of the invention is to provide a method for determining whether a woman is in the Niddah status.

According to a broad aspect of the present invention, there is provided a wiping device for wiping the surface of a tubular cavity or passageway, comprising: an implement having a distal end dimensioned and configured with a rounded tip to facilitate insertion of the device into the cavity; the implement also having a proximal end configured for manually grasping the implement and for inserting the distal end of the implement into the cavity; and a flexible wiping cloth configured to be received over the distal end of the implement, to conform to its rounded tip, and to define a lower edge to be releasable engaged by the implement in order to be releasably retained on the implement.

Two embodiments of the invention are described below for purposes of example.

In a first embodiment, the implement includes a top part having a distal end formed with said rounded tip, and a proximal end which is open; and a bottom part having a proximal end configured for manual grasping, and a distal end receivable within said proximal end of the top with said edge of the wiping cloth inbetween.

In another described embodiment, the implement includes an elongated shank; a handle at one end of the elongated shank graspable by a user for manually manipulating the implement; a head at the opposite end of the elongated shank dimensioned and configured for insertion into the cavity; and a retaining device for releasable retaining a wiping cloth on the outer surface of the head.

According to a yet further aspect of the invention, there is provided a method for determining whether a woman is in a Niddah status, comprising: applying a wiping cloth to the outer surface of an implement as described above; inserting the implement with the wiping cloth retained on its outer surface to wipe the surface of the vagina; removing the implement and wiping cloth from the vagina of the woman; removing the wiping cloth from the implement; and inspecting the wiping cloth for an indication whether the woman is in a Niddah status.

As will be described below, such an implement can be broadly used for conveniently and efficiently wiping surfaces of cavities or tubular passageways, and can be more particularly used by women for determining whether the woman is in the status of Niddah, especially where the assistance of a rabbi is needed or desired for this purpose. Such implements constructed in accordance with the present invention can be of a relatively simple construction constituted of a few simple parts which can be produced in volume and at low cost.

Further features and advantages of the invention will be apparent from the description below.

### BRIEF DESCRIPTION OF THE DRAWINGS

The invention is herein described, by way of example only, with reference to the accompanying drawings, wherein:

FIG. 1 illustrates one form of wiping device constructed in accordance with the present invention;

FIG. 2 is an exploded view illustrating the main parts of the wiping device of FIG. 1;

FIGS. 3a, 3b and 3c illustrate three examples of wiping cloths that may be used with the wiping device of FIGS. 1 and 2;

FIG. 4 is an enlarged side elevational view of the wiping device of FIG. 1;

FIG. 5 is a sectional view along line V-V of FIG. 4;

FIG. 6 is an enlarged fragmentary view of the encircled portion of FIG. 5;

FIG. 7 is an isometric view of the top part of the wiping device of FIGS. 1 and 2;

FIGS. 8, 9 and 10 are side, top and bottom views, respectively, of FIG. 7;

FIG. 11 is another view of the top part of the wiping tool;

FIGS. 12, 13 and 14 are sectional views along lines XII-XII, XIII-XIII and XIV-XIV of FIG. 11, respectively;

FIG. 15 is an isometric view of the bottom part of the wiping device of FIGS. 1 and 2;

FIGS. 16, 17 and 18 are side, top and bottom views, respectively, of FIG. 15;

FIG. 19 is another view of the bottom part of the tool of FIGS. 1 and 2;

FIGS. 20-23 are sectional views along lines XX, XXI, XXII and XXIII, respectively, of FIG. 19;

FIG. 24 illustrates another form of wiping device constructed in accordance with the invention, including the wiping cloth partly broken away and not attached to the implement;

FIG. 25 illustrates the implement of FIG. 24 with the wiping cloth attached to it;

FIG. 26 is an exploded view illustrating the main parts of the implement of FIGS. 24 and 25, without the wiping cloth;

FIG. 27 is an exploded view, corresponding to that of FIG. 26, but illustrating a modification in the construction of the implement;

FIG. 28 is a front elevational view of the shank and head of the implement of FIGS. 24 and 25;

FIG. 29 is a sectional view along line XXXI-XXXI of FIG. 28;

FIG. 30 is a transverse sectional view along line XXX-XXX of FIG. 28;

FIG. 31 is a front elevational view of the releasing device, namely a slidable sleeve, included in the implement of FIGS. 24 and 25;

FIG. 32 is a sectional view along line XXXI-XXXII of FIG. 31; and

FIG. 33 is an isometric view illustrating a variation in the construction of the slidable sleeve of FIG. 31.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS OF THE INVENTION

As indicated earlier, while the invention broadly involves a wiping implement for wiping the surfaces of a tubular passageway, it is particularly useful for determining whether a woman is in a Niddah status, and therefore the invention is described below particularly with respect to such an application.

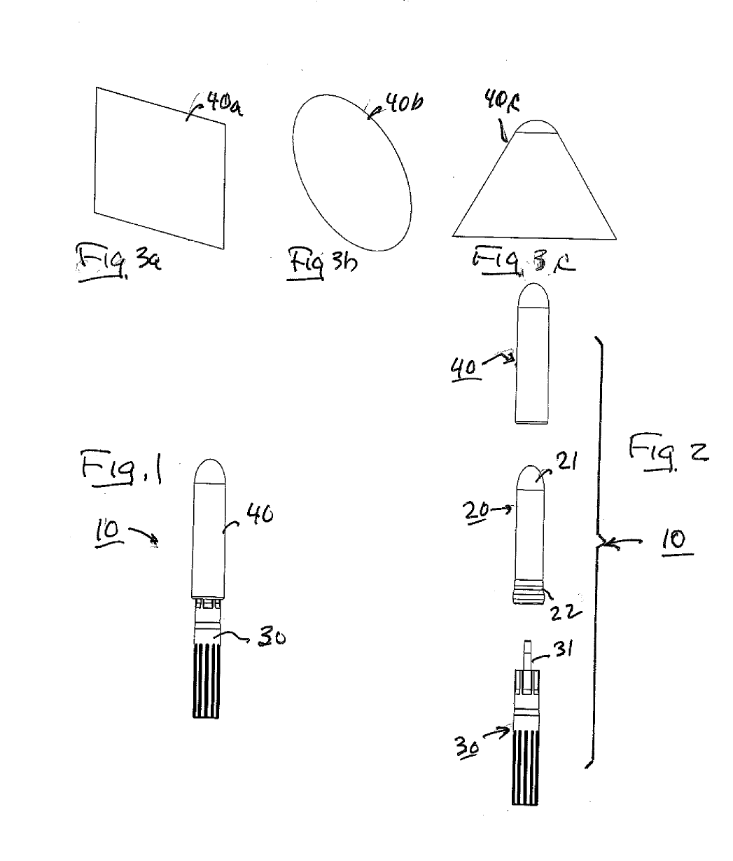

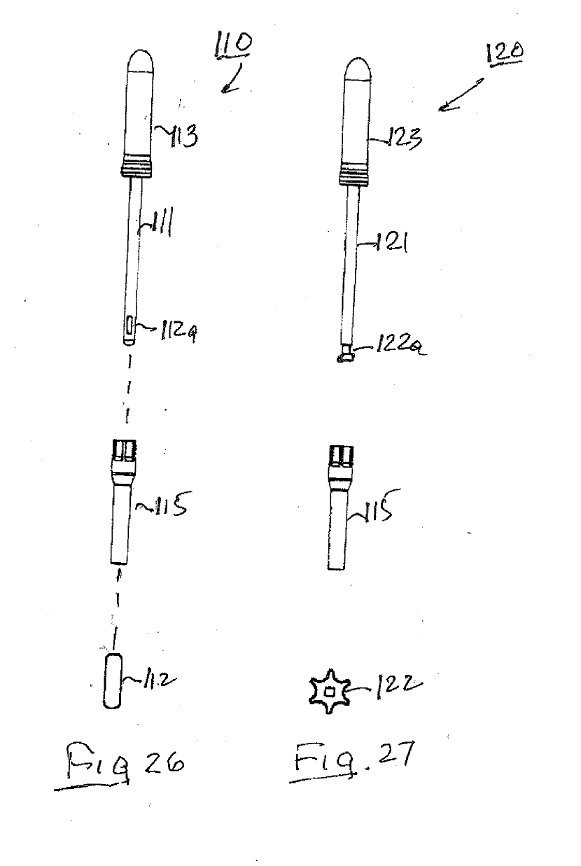

### The Embodiment of FIGS. 1-23

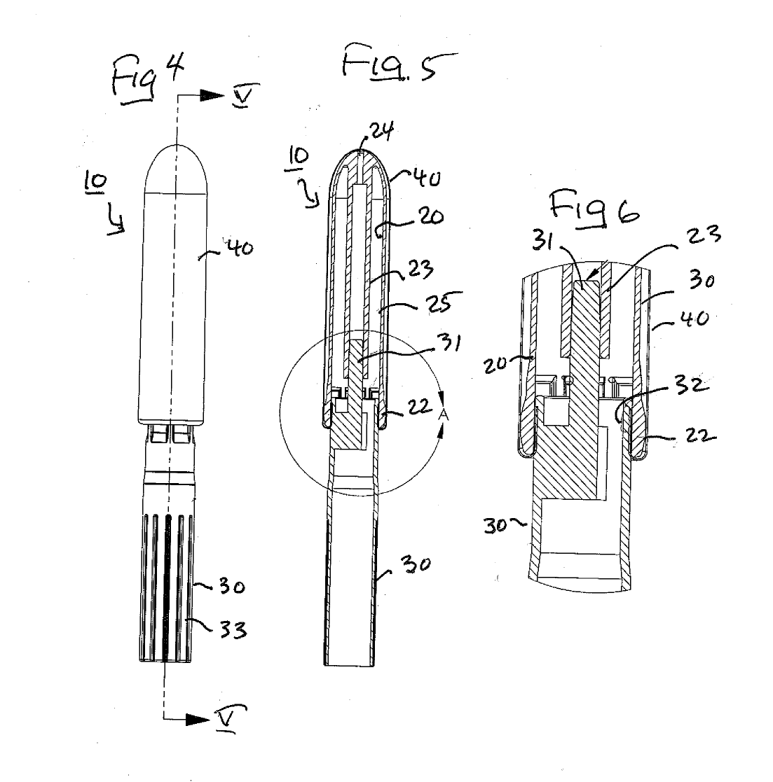

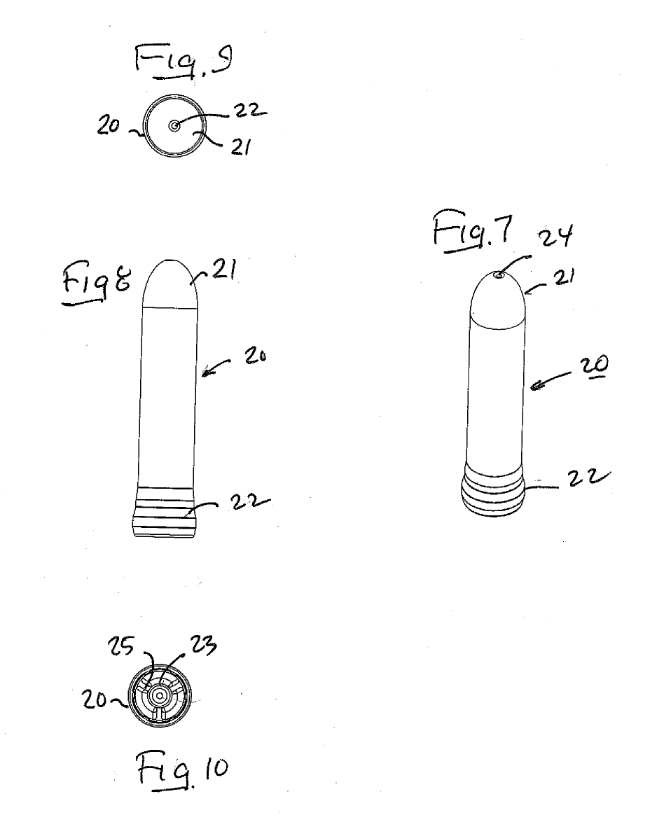

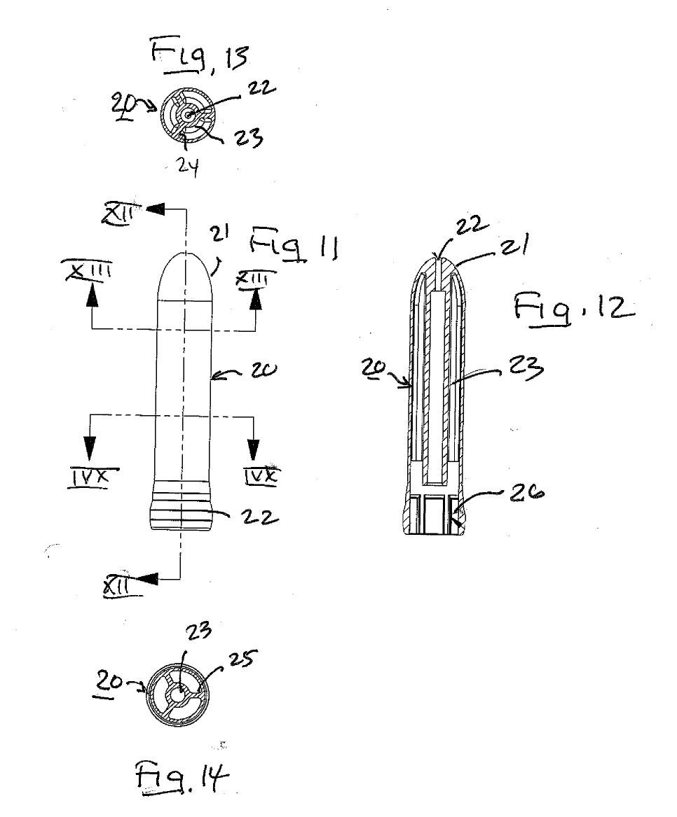

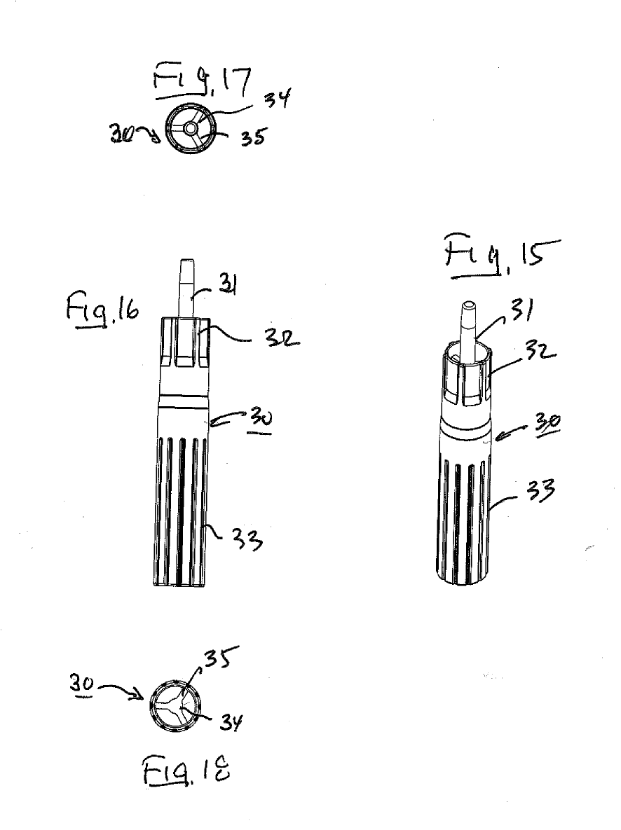

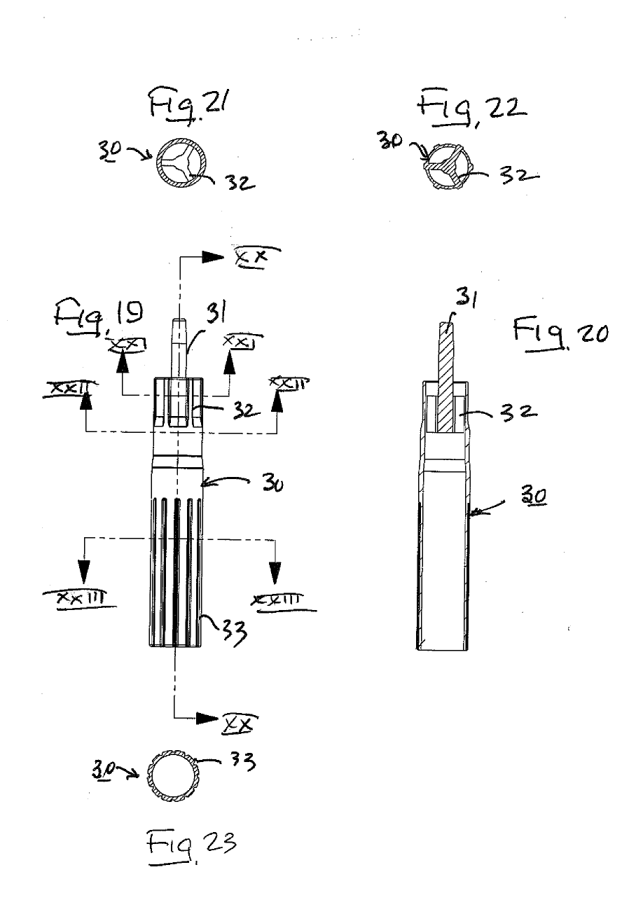

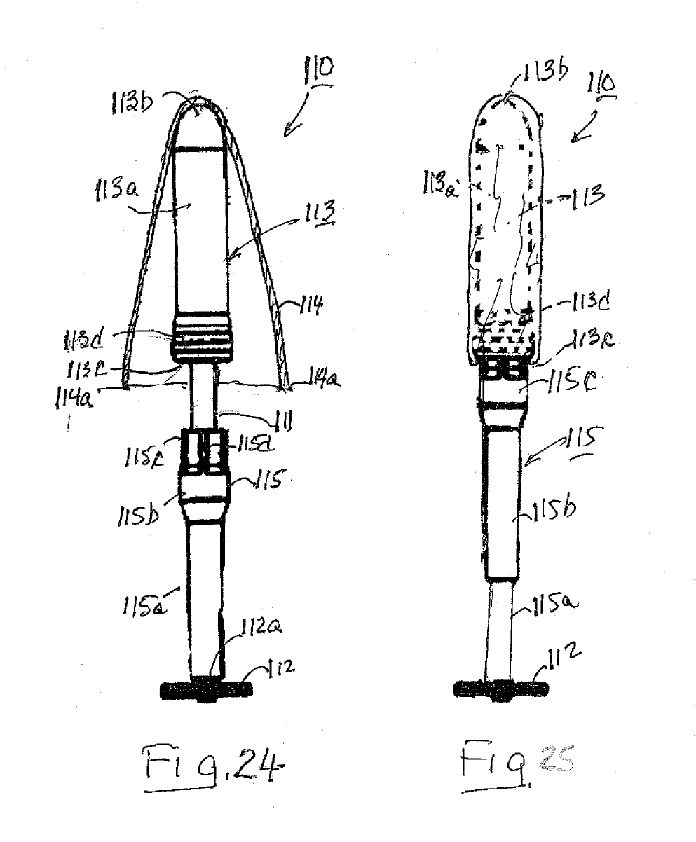

The wiping device illustrated in FIGS. 1-23, generally designated 10 in FIGS. 1 and 2, includes three main parts best seen in the exploded view of FIG. 2: a top part 20, a bottom part 30, and a wiping cloth 40. As will be described more particularly below, the top and bottom parts 20, 30, when assembled together, constitute the implement, and the wiping cloth 40 is received over, and releasably retained, on the top part 20 for use in wiping the cavity or longitudinal passageway, and then removed for inspecting the results of the wiping action.

The top part 20 has a distal end dimensioned and configured with a rounded tip 21 to facilitate insertion of the device into the cavity to be wiped. The proximal end of the top part 20 is formed with annular ribs 22 cooperable with the distal end of the bottom part 30 for releasably retaining the wiping cloth 40 on the top part. As shown particularly in FIGS. 5 and 6, the top part 20 further includes a hollow stem 23 open at the bottom (proximal end) and extending longitudinally through the top part to the opposite end (distal end) where it terminates in a small-diameter opening 24. Hollow stem 23 is supported by three radially-extending ribs 35 (FIG. 18) integrally formed with the hollow stem. Stem 23 receives a pin 31 at the distal end of the bottom part 30 for releasable attaching the two parts 20 and 30 together, with the lower edge of the wiping cloth 40 wedged inbetween, while opening 24 serves to vent the interior of stem 23 to the atmosphere during the attachment and detachment of the two parts.

The bottom part 30 is more particularly illustrated in FIGS. 15-18. It includes, in addition to a plurality of longitudinally-extending slits 32 at its distal end to increase the flexibility of the part for gripping the proximal end of the top part 20, with the wiping cloth 40 inbetween. In addition, the proximal end of the bottom part 30 is formed with a plurality of longitudinally-extending ribs 33 which serve to facilitate manually grasping the proximal end of the bottom part 30. Ribs 33 also constitute the proximal end of the implement.

The bottom part 30 is reinforced by a core 34 (FIG. 18) consisting of three radially-extending ribs 35.

FIGS. 3a, 3b and 3c illustrate, for purposes of example, three configurations of the wiping cloth 40 shown in FIG. 2. Thus, the wiping cloth 40a illustrated in FIG. 3a, is initially of a square configuration. Its central region is to be placed over the rounded tip 21 of the top part 20 of the implement, and wrapped around the top part such that its lower edges extend just past the ribs 22 of the top part 20 such that when the bottom part 30 is inserted into the end of the top part 20, the lower edges of the wiping cloth are wedged between the slitted end 32 of the bottom part 30, as best seen in FIG. 6.

In FIG. 3b, the wiping cloth, therein shown at 40b, is initially of a circular configuration, and is applied over the top part 20 of the implement, in the same manner as shown in FIG. 6. In FIG. 3c the wiping cloth is initially of a conical configuration, and is applied in the same manner as discussed above with respect to FIGS. 3a and 3b.

The manner of using the implement of FIGS. 1-23 for the determining whether a woman is in a Niddah status will be apparent from the above description.

Thus, the wiping cloth 40 is first applied over the top part 20 of the implement, and then the bottom part 30 is applied to the top part 20, such as to wedge the lower edges of the wiping cloth between the two parts, as described above particularly with respect to FIG. 6. The implement may then be inserted into the woman's vagina, rotated so as to pick up any fluids therein, and then removed. The wiping cloth 40 may then be detached from the implement by merely withdrawing the bottom part 30 from the top part 20, to enable the wiping cloth to be visually inspected.

### The Embodiment of FIGS. 24-33

FIGS. 24-33 illustrate another wiping device, therein generally designated 110, constructed in accordance with the invention. It includes an elongated shank 111 carrying a handle 112 at one end (the proximal end) which is graspable by a user for manually manipulating the implement, and a head 113 at the opposite end (the distal end) for insertion into the cavity or passageway to be wiped, in this case the woman's vagina. Before the head 113 is inserted, it is draped with a wiping cloth 114, which may be of any suitable configuration, such as circular or rectangular. The wiping cloth is of a length to extend past the head 113, towards the handle 112, for releasable attachment to the head as described above with respect to FIG. 6.

As shown particularly in FIG. 29, the head 113 includes a main body 113a of hollow cylindrical configuration terminating at the distal end in a rounded tip 113b, preferably of semi-circular configuration, and open at its proximal end 113c for receiving the elongated shank 111. The proximal end of head 113, namely that facing opposite to its rounded tip 113a, is formed with a plurality of annular ribs 113b in order to better retain the wiping cloth 114 on the head during its use.

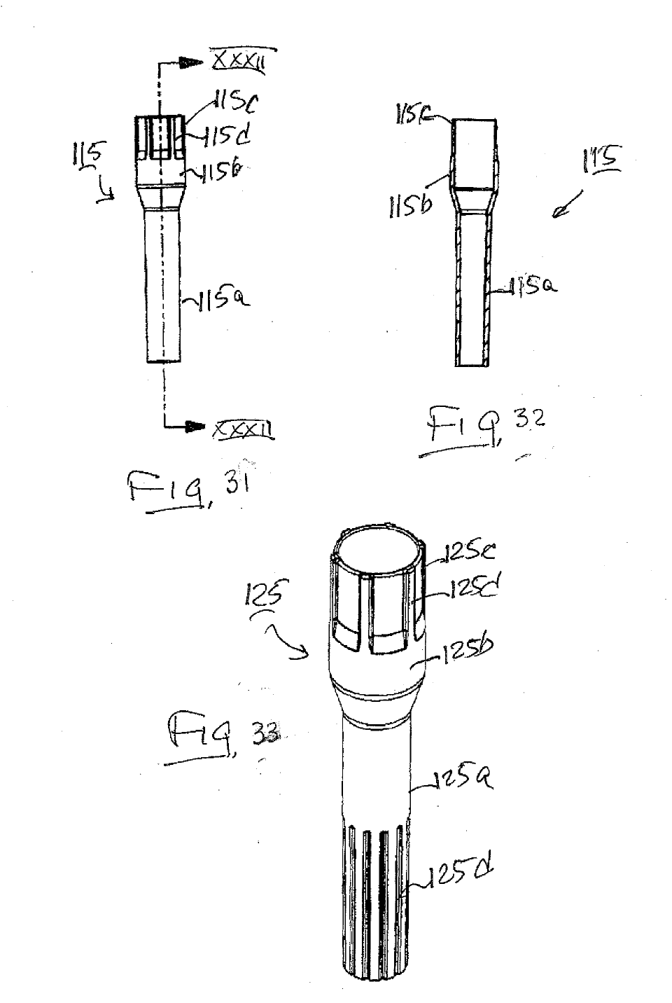

As shown particularly in FIG. 30, the shank 111 is of circular cross-section and is received within the open end 113c of the head 113. The complementary surfaces of the head 113 and the elongated shank 11 may be formed with slots or keys to non-rotatably couple the head to the shank, such that the head is rotated with the shank when the shank is rotated.

The construction of the slidable sleeve 115, which as indicated above serves as a retaining device for releasably retaining the wiping cloth 114 on the outer surface of the head 113, is best seen in FIGS. 31 and 32. It includes a lower section 115a of hollow cylindrical configuration for enclosing the shank 11, an upper section 115d of larger diameter, and an upper end 115c which, as best seen in FIG. 32, is formed with a decreasing wall thickness towards the end facing the head. This facilitates the insertion of the sleeve within the open end of the head for releasably wedging the open end (114b, FIG. 24) of the wiping cloth 114 between the outer edges of the head 113 and the outer surface of sleeve section 115c.

In addition, the outer tapered end (distal end) of section 115c is formed with a plurality of axially-extending slits 115d to define fingers 115e which enhance the flexibility of that end of the sleeve. The latter end is received within the open end (proximal end) 113c of head 113, with the outer edges 114b of the wiping cloth 114 wedged inbetween, as described above with respect to FIG. 6.

The manner of using the wiping device of FIGS. 24-33 for determining whether a woman is in a Niddah status will be apparent from the above description. Thus, with the slidable sleeve 115 in its lowermost position as illustrated in FIG. 24, a wiping cloth 114 is applied over the rounded end 113a of the head 113 such that the lower edges 114b of the wiping cloth extend past the lower end 113c of the head. While the lower edges 114b of the wiping cloth 114 are pressed against the elongated shank 11, sleeve 115 is slid upwardly such that its tapered, slitted upper wall 115c enters the lower edges 113c of the head, firmly wedging the lower end 114b of the wiping cloth 114 between the inner surface of head 113 and the outer surface of sleeve section 115c. This is the condition of the implement illustrated in FIG. 25.

The implement may now be inserted into the woman's vagina, rotated so as to pick-up any fluids therein, and removed. Sleeve 115 may then be moved to its releasing position, as illustrated in FIG. 24, thereby releasing the wiping cloth 114 from the implement. This enables the implement to be used with another wiping cloth, while the removed wiping cloth can be examined for determining whether or not the woman is the Niddah status.

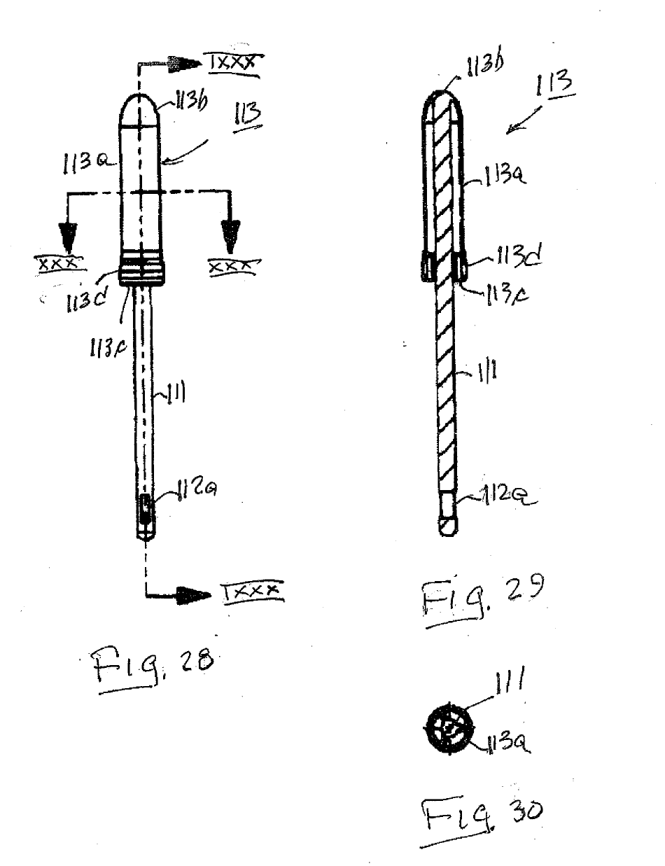

FIG. 27 is similar to FIG. 26, but illustrates a variation in the construction of the implement. Thus, the implement illustrated in FIG. 27, and therein generally designed 120, includes an elongated shank 121, a handle 122 at the proximal end of the implement, a head 123 at the distal end, and a slidable sleeve 125, similar to the construction of the corresponding elements in FIG. 26, except that the handle 122 is in the form of a ribbed disk 122 or the like which is snapped into an annular groove 122a formed at the respective end of the shank 121. In all other respects, the implement illustrated in FIG. 27 is of the same construction, and is used in the same manner, as described above.

FIG. 33 illustrates a further modification that may be included in the construction of the slidable sleeve. In this variation, the slidable sleeve, therein generally designated 125, is formed on its outer surface at its lower end (proximal end) with a plurality of longitudinally-extending ribs 25e to facilitate the manipulation of the sleeve to its releasing position (FIG. 24) or to its securing position (FIG. 25). In all other respect, sleeve 125 illustrated in FIG. 33 is of the same construction as described above, including a lower (proximal) cylindrical section 125a, an intermediate section 125b of larger diameter, and an upper (distal) tapered section 25c formed with a plurality of slits 25d to facilitate its insertion into the lower (proximal) open end 113c of head 113, with the lower end 114b of the wiping cloth 114 wedged inbetween.

While the invention has been described with respect to one preferred embodiment, with several modifications, and with respect to one preferred application, it will be appreciated that these are set forth for purposes of example, and that many other variations, modifications and applications of the invention may be made.

What is claimed is:

1. A wiping device for wiping the surface of a cavity, comprising:
an implement having a distal end dimensioned and configured with a rounded tip to facilitate insertion of the device into the cavity;
said implement also having a proximal end configured for manually grasping the implement and for inserting the distal end of the implement into said cavity;
and a flexible wiping cloth configured to be received over said distal end of the implement, to conform to its rounded tip, and to define a lower edge to be releasable engaged by said implement in order to be releasably retained on the implement.

2. The wiping device according to claim 1, wherein said implement includes:
a top part having a distal end formed with said rounded tip, and a proximal end which is open;
and a bottom part having a proximal end configured for manual grasping, and a distal end receivable within said proximal end of the top with said edge of the wiping cloth inbetween.

3. The wiping device according to claim 2, wherein the rounded tip of the top part includes an opening for venting to the atmosphere air within the top part when receiving the bottom part.

4. The wiping device according to claim 2, wherein the top part includes an internal tubular stem, and the bottom part includes a pin telescopically received within said tubular stem for releasably retaining the top and bottom parts together with the lower edge of the wiping cloth inbetween.

5. The wiping device according to claim 2, wherein the distal end of said bottom part is formed with ribs for engaging the lower edge of the wiping cloth to releasably retain it between said ribs and the proximal open end of said top part.

6. The wiping device according to claim 5, wherein the proximal end of said bottom part is also formed with ribs for facilitating grasping and manipulating the implement.

7. The wiping device according to claim 1, wherein said distal end is of substantially cylindrical configuration and includes an outer tip of semi-spherical configuration to facilitate its insertion into said cavity.

8. The wiping device according to claim 1, wherein said implement includes:
an elongated shank;
a handle at one end of said elongated shank graspable by a user for manually manipulating the implement;
a head at the opposite end of the elongated shank dimensioned and configured for insertion into the cavity;
and a retaining device for releasable retaining the wiping cloth on the outer surface of said head.

9. The implement according to claim 1, wherein said head has an outer tip of rounded configuration to facilitate its insertion into said passageway.

10. The wiping device according to claim 8, wherein said retaining device includes a sleeve slidable on said shank towards said open end of the head for securing the wiping cloth thereto, or away from said open end of the head for releasing the wiping cloth therefrom.

11. The wiping device according to claim 8, wherein said open end of the head is formed with a plurality of annular ribs on its outer surface to enhance its gripping of the wiping cloth when the slidable sleeve is slid into the open end of the head.

12. The wiping device according to claim 10, wherein said sleeve is formed with a wall of decreasing thickness towards the end facing the head to facilitate the insertion of said end of the sleeve into the open end of the head for releasable retaining the wiping cloth on the outer surface of the head.

13. The wiping device according to claim 10, wherein the end of said sleeve facing the head is formed with longitudinally-extending slits to increase its flexibility, and thereby to facilitate its insertion into the open end of the head for releasable retaining the wiping cloth on the outer surface of said head.

14. A wiping device for wiping the vagina of a Jewish woman to determine whether the woman is in a Niddah status, comprising:
an implement having a distal end dimensioned and configured with a rounded tip to facilitate insertion of the device into the vagina;
said implement also having a proximal end configured for manually grasping the implement and for inserting the distal end of the implement into said vagina;
and a flexible wiping cloth configured to be received over said distal end of the implement, to conform to its rounded tip, and to define a lower edge to be releasable engaged by said implement in order to be releasably retained on the implement.

15. The wiping device according to claim 14, wherein said implement includes:
a top part having a distal end formed with said rounded tip, and a proximal end which is open;
and a bottom part having a proximal end configured for manual grasping, and a distal end receivable within said proximal end of the top with said edge of the wiping cloth inbetween.

16. The wiping device according to claim 15, wherein the rounded tip of the top part includes an opening for venting to the atmosphere air within the top part when receiving the bottom part.

17. The wiping device according to claim 14, wherein said implement includes:
an elongated shank;
a handle at one end of said elongated shank graspable by a user for manually manipulating the implement;
a head at the opposite end of the elongated shank dimensioned and configured for insertion into the vagina;
and a retaining device for releasable retaining the wiping cloth on the outer surface of said head.

18. The implement according to claim 17, wherein said head has an outer tip of rounded configuration to facilitate its insertion into said passageway.

19. A method for determining whether a woman is in a Niddah status, comprising:
applying a wiping cloth to the outer surface of an implement according to claim 1;
inserting the implement with the wiping cloth retained on its outer surface to wipe the surface of the vagina;
removing the implement and wiping cloth from the vagina of the woman;
removing the wiping cloth from the implement;
and inspecting the wiping cloth for an indication whether the woman is in a Niddah status.
